(19) United States
(12) Patent Application Publication
(10) Pub. No.: US 2019/0371470 A1
(43) Pub. Date: Dec. 5, 2019

(54) METHOD OF PREDICTING RISK TREATING CRITICAL LIMB ISCHEMIA WITH CONCENTRATED BONE MARROW NUCLEATED CELLS

(71) Applicants: Hillary Overholser, Goshen, IN (US); James M. McKale, Cincinnati, OH (US); Michael G. Wilson, Warsaw, IN (US)

(72) Inventors: Hillary Overholser, Goshen, IN (US); James M. McKale, Cincinnati, OH (US); Michael G. Wilson, Warsaw, IN (US)

(21) Appl. No.: 16/429,947

(22) Filed: Jun. 3, 2019

Related U.S. Application Data
(60) Provisional application No. 62/681,030, filed on Jun. 5, 2018.

(51) Int. Cl. G16H 50/30; G16H 50/70

(52) U.S. Cl. CPC G16H 50/30; G16H 50/20; G16H 50/70

(57) ABSTRACT

Methods and systems for determining risk of treating a patient suffering from critical limb ischemia with concentrated bone marrow nucleated cells. For example, a machine-implemented method can include calculating a patient risk score from one or more value each corresponding to a characteristic of the patient, wherein each value is determined and weighted according to influence of the corresponding characteristic on patient outcome in a population of patients suffering from critical limb ischemia treated with concentrated bone marrow nucleated cells; comparing the calculated patient risk score to a pre-determined risk score corresponding to the population of patients; and determining from the compared risk scores the risk of treating a patient suffering from critical limb ischemia with concentrated bone marrow nucleated cells. This result can be used to determining an appropriate treatment plan for a patient and the methods described herein may further comprise treating the patient accordingly.

### CLAIM OF PRIORITY

This application claims the benefit of U.S. Provisional Patent Application Ser. No. 62/681,030, filed on Jun. 5, 2018, the benefit of priority of which is claimed hereby, and which is incorporated by reference herein in its entirety.

### BACKGROUND

Peripheral arterial disease (PAD) is a widespread disease that primarily effects older populations. PAD results from narrowing of arteries and reduction of blood flow. Left untreated, PAD may progress to critical limb ischemia (CLI) which may be characterized by pain, numbness or tissue loss in the extremities due to arterial insufficiency. A diagnosis of CLI represents a serious risk of amputation.

Cell therapy is currently being investigated as a potential therapeutic option for CLI. For example, intramuscular injection of bone marrow aspirate harvested and concentrated via MarrowStim™ may decrease major amputations in some patients. (Wang et al., J Vase Surg. 2017). Concentrated bone marrow nucleated cells (cBMNC) represent a promising therapeutic development. However, there is currently no quantitative method to assess the likelihood of success of any cellular therapy for CLI.

### OVERVIEW

This disclosure pertains generally to machine implemented methods and systems for determining risk of failure of treating a subject suffering from critical limb ischemia with concentrated bone marrow nucleated cells.

Although a given treatment may be shown safe and efficacious in a general population, predicting the risk or benefit to a specific patient can be challenging. The present inventors have recognized, among other things, that certain patient population subgroups suffering from CLI have a different risk-benefit profile than other subgroups.

Example machine implemented methods for determining a patient specific, or patient subgroup specific, risk of failure of treating a subject suffering from critical limb ischemia with concentrated bone marrow nucleated cells are described.

Based at least in part on the patient-specific soft tissue location, the example systems and methods can also be utilized in determining an appropriate treatment plan for a patient suffering from CLI. In some examples, the treatment plan may be intramuscular administration of autologous concentrated bone marrow nucleated cells harvested and concentrated via a MarrowStim™ device.

In the following description, for purposes of explanation, numerous specific details are set forth in order to provide a thorough understanding of examples provided. It will be evident, however, to one skilled in the art that examples of the present invention may be practiced without these specific details or details may be modified to a degree. It will also be evident that the systems and methods discussed are not limited to the examples provided and may include other scenarios not specifically discussed.

For example, the methodologies discussed herein with respect to use of a MarrowStim™ device may be similarly applied to other devices or other similar procedures (e.g., concentrated bone marrow nucleated cells harvested and concentrated by other means or concentrated bone marrow nucleated cells administered to the patient via a different mode of administration).

To further illustrate the methods and systems disclosed herein, a non-limiting list of examples is provided here:

In Example 1, a method for determining risk of treating a patient suffering from critical limb ischemia with concentrated bone marrow nucleated cells, comprising:

calculating a patient risk score from one or more value each corresponding to a characteristic of the patient, wherein
each value is determined and weighted according to influence of the corresponding characteristic on patient outcome in a population of patients suffering from critical limb ischemia treated with concentrated bone marrow nucleated cells;

comparing the calculated patient risk score to a pre-determined risk score corresponding to the population of patients; and

determining from the compared risk scores the risk of treating a patient suffering from critical limb ischemia with concentrated bone marrow nucleated cells.

In Example 2, the method of Example 1, wherein each characteristic is, individually, a demographic characteristic, a disease history, a symptom, or any combination thereof.

In Example 3, the method of any one or combination of Examples 1-2, wherein each characteristic is, individually, a history of diabetes, a history of kidney disease, history of stroke, history of transient ischemic attack, history of myocardial infarction, history of cardiac arrhythmia, history of congestive heart failure, history of DVT, a history of amputation, Rutherford category, sex, ethnicity, age, HbA1c status, ambulatory status, number of wounds, size of wounds, or any combination thereof.

In Example 4, the method of any one or combination of Examples 1-3, wherein at least one value corresponds to a characteristic which is a history of diabetes, a history of renal disease, Rutherford score, or age.

In Example 5, the method of any one or combination of Examples 1-4, wherein the patient risk score is calculated, at least in part, based on the patient age.

In Example 6, the method of any one or combination of Examples 1-5, wherein the patient risk score is calculated, at least in part, based on the patient history of diabetes.

In Example 7, the method of any one or combination of Examples 1-6, wherein the patient risk score is calculated, at least in part, based on the patient history of renal disease.

In Example 8, the method of any one or combination of Examples 1-7, wherein the patient risk score is calculated, at least in part, based on the patient atherosclerotic disease burden.

In Example 9, the method of any one or combination of Examples 1-8, wherein each value is a coefficient determined by multivariate analysis of a population of patients suffering from critical limb ischemia treated with concentrated bone marrow nucleated cells.

In Example 10, the method of any one or combination of Examples 1-9, wherein patient outcome is quantified in terms of time to amputation or death.

In Example 11, the method of any one or combination of Examples 1-10, wherein the concentrated bone marrow nucleated cells are autologous cells harvested and concentrated via a MarrowStim device.

In Example 12, the method of any one or combination of Examples 1-11, further comprising treating the patient with a treatment expected to have the same or lower risk compared to the determined risk.

In Example 13, the method of any one or combination of Examples 1-12, further comprising treating the patient with the concentrated bone marrow nucleated cells.

In Example 14, a system, comprising:

a computer including at least one processor and a memory device, the memory device including instructions that, when executed by the at least one processor, cause the computer to:
access a database containing one or more value each corresponding to an inputted characteristic of a patient suffering from critical limb ischemia, wherein each value is determined and weighted according to influence of the corresponding characteristic on patient outcome in a population of patients suffering from critical limb ischemia treated with concentrated bone marrow nucleated cells;
calculate from the one or more values a patient risk score;
compare the calculated patient risk score to a pre-determined risk score corresponding to the population of patients;
determine based on the compared risk scores the risk of treating a patient suffering from critical limb ischemia with concentrated bone marrow nucleated cell.

In Example 15, a machine-readable storage device including instructions that, when executed by a machine, cause the machine to:

access a database containing one or more value each corresponding to an inputted characteristic of a patient suffering from critical limb ischemia, wherein each value is determined and weighted according to influence of the corresponding characteristic on patient outcome in a population of patients suffering from critical limb ischemia treated with concentrated bone marrow nucleated cells;

calculate from the one or more values a patient risk score;

compare the calculated patient risk score to a pre-determined risk score corresponding to the population of patients;

determine based on the compared risk scores the risk of treating a patient suffering from critical limb ischemia with concentrated bone marrow nucleated cell.

In Example 16, a machine-implemented method including:
inputting a plurality of characteristics of a patient suffering from critical limb ischemia as data into a computer;
accessing a database stored in a memory device module containing a plurality of values each corresponding to a patient characteristic, wherein each value is derived from previously-stored patient outcome data for a population of patients suffering from critical limb ischemia treated with concentrated bone marrow nucleated cells and each value represents weighted influence of the corresponding characteristic on patient outcome in the population;
referencing each value corresponding to each of the inputted characteristics;
calculating a patient risk score from the referenced values;
comparing the calculated patient risk score to a threshold risk score from the known patient outcome;
determining based on the comparison a risk of treating a patient suffering from critical limb ischemia with concentrated bone marrow nucleated cells; and
outputting the result of the determining to a user.

In Example 17, the system or method of any one or any combination of Examples 1-16 can optionally be configured such that all elements or options recited are available to use or select from.

These and other examples and features of the present systems and methods will be set forth in part in the following Detailed Description. This Overview is intended to provide non-limiting examples of the present subject matter—it is not intended to provide an exclusive or exhaustive explanation. The Detailed Description below is included to provide further information about the present systems and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various examples discussed in the present document.

FIGS. 1A and 1B show a summary of demographical information all treated subjects in the Continued Access and Pivotal Study of obtaining cBMNC using MarrowStim™ kit and administering to a patient suffering from CLI.

FIG. 2 shows results (time to event) for using cBMNC to treat CLI for all treated subjects in the combined studies, adjusted and unadjusted based on age, and showing treatment effect with and without covariate.

FIG. 3 shows results (time to event) for using cBMNC to treat CLI for interventional group subjects in the combined studies, adjusted and unadjusted based on age, showing treatment effect with and without covariate.

FIG. 4 shows results (time to event) for using cBMNC to treat CLI in all randomized subjects in the combined studies, examining treatment effect based on age with an age cutoff of 70, and showing treatment effect interaction based on a model having covariates to treatment, Rutherford severity and diabetes.

FIG. 5 shows results (time to event) for using cBMNC to treat CLI in subjects with age<=70 in the combined studies, without covariate.

FIG. 6 shows results (time to event) for using cBMNC to treat CLI in subjects with age>70 in the combined studies, without covariate.

FIG. 7 shows results (time to event) for using cBMNC to treat CLI in all randomized subjects in the combined studies, examining treatment effect based on age with an age cutoff of 80, and showing treatment effect interaction based on a model having covariates to treatment, Rutherford severity and diabetes.

FIG. 8 shows results (time to event) for using cBMNC to treat CLI in subjects with age<=80 in the combined studies, without covariate.

FIG. 9 shows results (time to event) for using cBMNC to treat CLI in subjects with age>80 in the combined studies, without covariate.

FIG. 10 shows results (time to event) for using cBMNC to treat CLI in all randomized subjects in the combined studies, examining treatment effect based on gender, and showing treatment effect interaction based on a model having covariates to treatment, Rutherford severity and diabetes.

FIG. 11 shows results (time to event) for using cBMNC to treat CLI in male subjects in the combined studies, without covariate.

FIG. 12 shows results (time to event) for using cBMNC to treat CLI in female subjects in the combined studies, without covariate.

FIG. 13 shows results (time to event) for using cBMNC to treat CLI in all randomized subjects in the combined studies, examining treatment effect based on ethnicity, and showing treatment effect interaction based on a model having covariates to treatment, Rutherford severity and diabetes.

FIG. 14 shows results (time to event) for using cBMNC to treat CLI in white non-Hispanic subjects in the combined studies, without covariate.

FIG. 15 shows results (time to event) for using cBMNC to treat CLI in subjects having ethnicity other than white-non hispanic in the combined studies, without covariate.

FIG. 16 shows results (time to event) for using cBMNC to treat CLI in all randomized subjects having diabetes in the combined studies, examining treatment effect based on HBA1C, and showing treatment effect interaction based on a model having covariates to treatment and Rutherford severity.

FIG. 17 shows results (time to event) for using cBMNC to treat CLI in subjects having diabetes and HBA1C<=8 in the combined studies, without covariate.

FIG. 18 shows results (time to event) for using cBMNC to treat CLI in subjects having diabetes and HBA1C>8 in the combined studies, without covariate.

FIG. 19 shows results (time to event) for using cBMNC to treat CLI in all randomized subjects in the combined studies, examining treatment effect based on renal disease, and showing treatment effect interaction based on a model having covariates to treatment and Rutherford severity.

FIG. 20 shows results (time to event) for using cBMNC to treat CLI in subjects without renal disease in the combined studies, without covariate.

FIG. 21 shows results (time to event) for using cBMNC to treat CLI in subjects with renal disease in the combined studies, without covariate.

FIG. 22 shows results (time to event) for using cBMNC to treat CLI in all randomized subjects in the combined studies, examining treatment effect based on BMI, and showing treatment effect interaction based on a model having covariates to treatment and Rutherford severity.

FIG. 23 shows results (time to event) for using cBMNC to treat CLI in subjects with BMI<=27.25 in the combined studies, without covariate.

FIG. 24 shows results (time to event) for using cBMNC to treat CLI in subjects with BMI>27.25 in the combined studies, without covariate.

FIG. 25 shows results (time to event) for using cBMNC to treat CLI in all randomized subjects in the one-year pivotal study, examining treatment effect based on site, diabetes, and Rutherford severity, and showing treatment effect interaction based on a model having covariates to treatment, Rutherford severity, diabetes, and site.

FIG. 26 shows results (time to event) for using cBMNC to treat CLI in all randomized subjects in the one-year pivotal study, examining treatment effect based on smoking, and showing treatment effect interaction based on a model having covariates to treatment.

FIG. 27 shows results (time to event) for using cBMNC to treat CLI in all randomized subjects in the one-year pivotal study, examining treatment effect based on body weight, and showing treatment effect interaction based on a model having covariates to treatment.

FIG. 28 shows results (time to event) for using cBMNC to treat CLI in all randomized subjects in the one-year pivotal study, examining treatment effect based on BMI, and showing treatment effect interaction based on a model having covariates to treatment.

FIG. 29 shows results (time to event) for using cBMNC to treat CLI in all randomized subjects in the one-year pivotal study, examining treatment effect based on ethnicity, and showing treatment effect interaction based on a model having covariates to treatment.

FIG. 30 shows results (time to event) for using cBMNC to treat CLI in all randomized subjects in the one-year pivotal study, examining treatment effect based on ethnicity (combining african americans, hispanics and other), and showing treatment effect interaction based on a model having covariates to treatment, Rutherford severity, diabetes and site.

FIG. 31 shows results (time to event) for using cBMNC to treat CLI in all randomized subjects in the one-year pivotal study, examining treatment effect based on gender, and showing treatment effect interaction based on a model having covariates to treatment, Rutherford severity, diabetes and site.

FIG. 32 shows results (time to event) for using cBMNC to treat CLI in all randomized subjects in the one-year pivotal study, examining treatment effect based on male gender, and showing treatment effect interaction based on a model having covariates to treatment, Rutherford severity, diabetes and site, and an analysis of maximum likelihood estimates.

FIG. 33 shows results (time to event) for using cBMNC to treat CLI in all randomized subjects in the one-year pivotal study, examining treatment effect based on female gender, and showing treatment effect interaction based on a model having covariates to treatment, Rutherford severity, diabetes and site.

FIG. 34 shows results (time to event) for using cBMNC to treat CLI in all randomized subjects in the one-year pivotal study, examining treatment effect based on gender, and showing treatment effect interaction based on a reduced model having covariates to treatment, Rutherford severity, diabetes and site.

### DETAILED DESCRIPTION

Example methods and systems for determining risk of failure of treating a subject suffering from critical limb ischemia (CLI) with concentrated bone marrow nucleated cells (cBMNC) are described. The example methods can also be utilized in determining an appropriate treatment plan for a patient and may further comprise treating the patient.

The method may include, for example, calculating a patient risk score from one or more value each corresponding to a characteristic of the patient suffering from CLI.

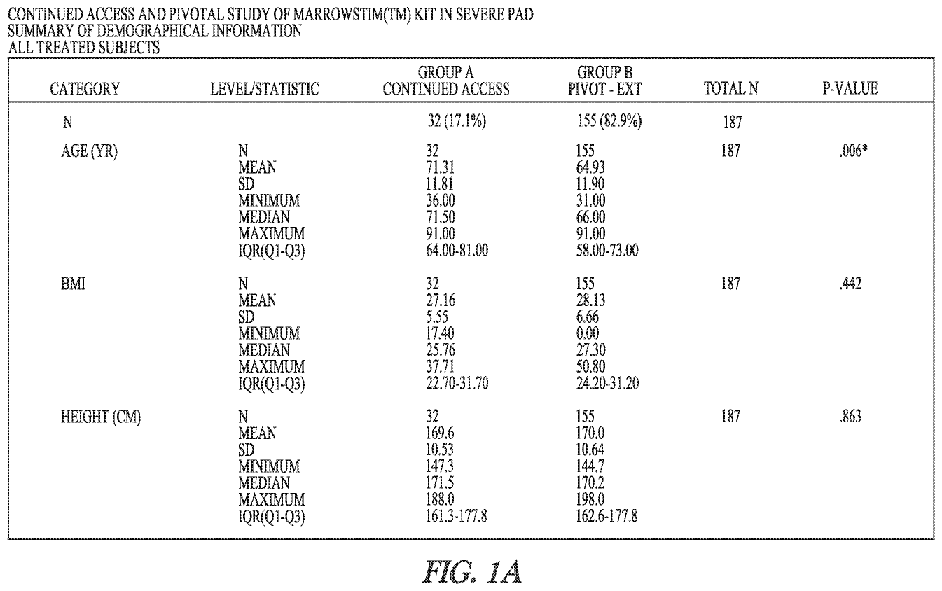

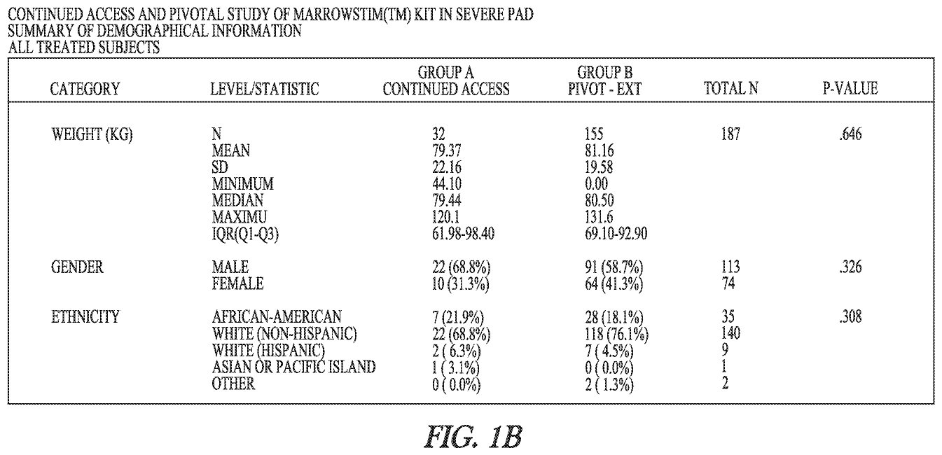

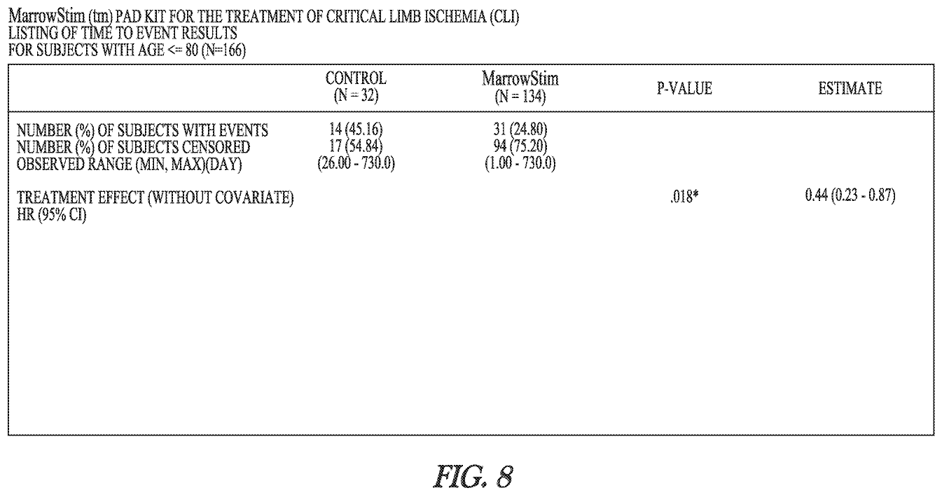

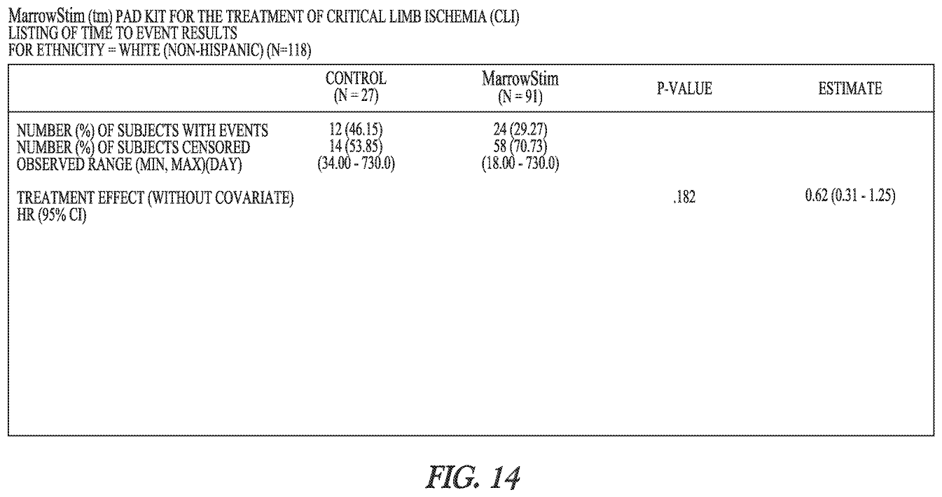

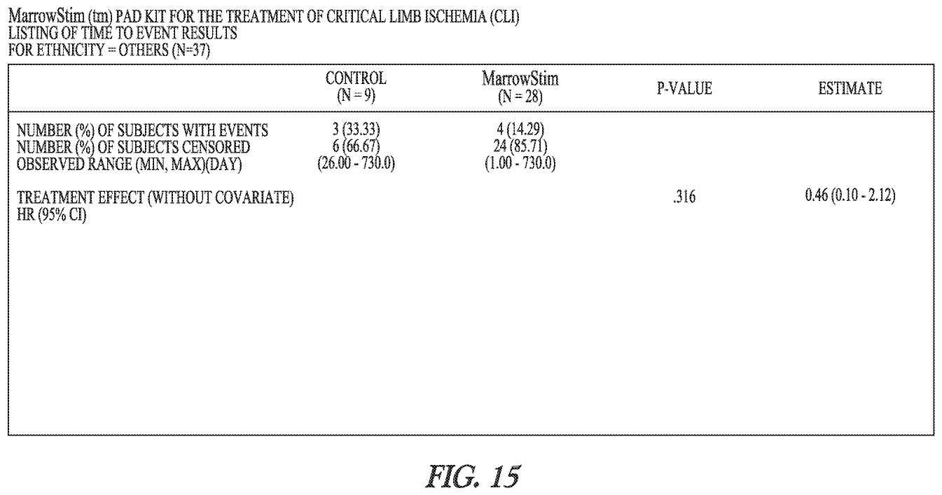

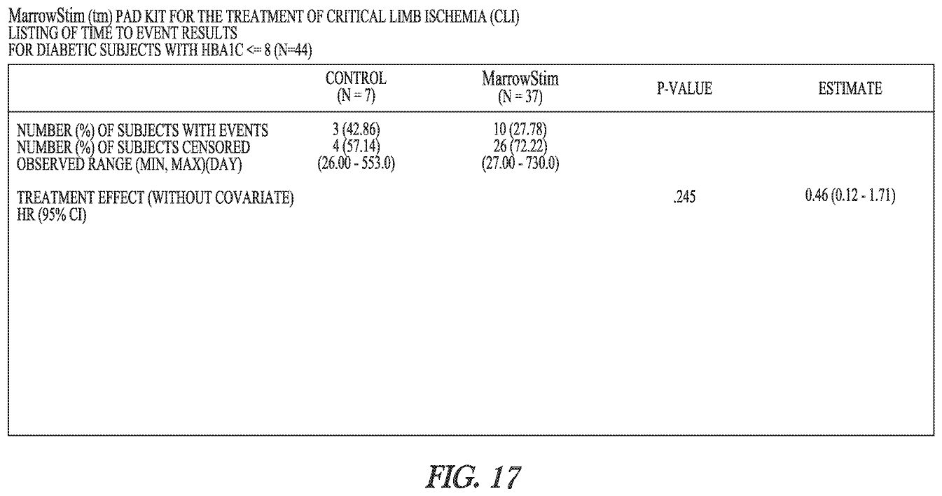

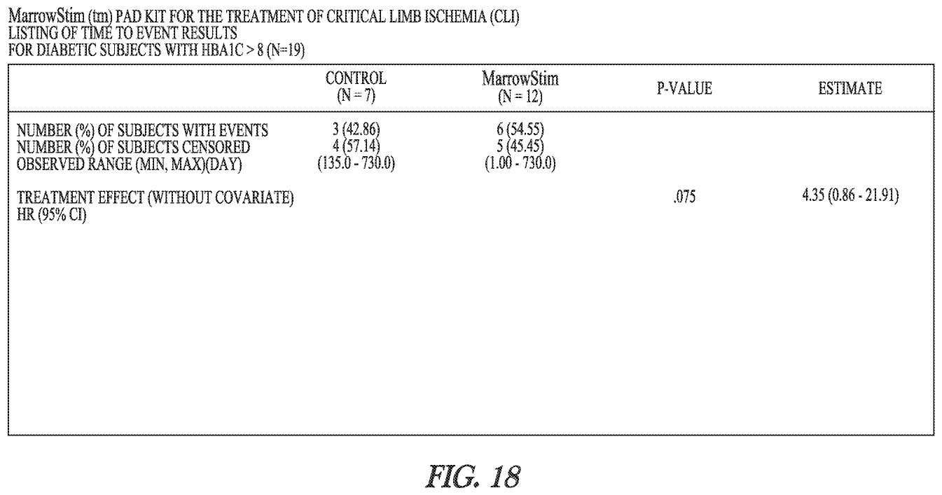

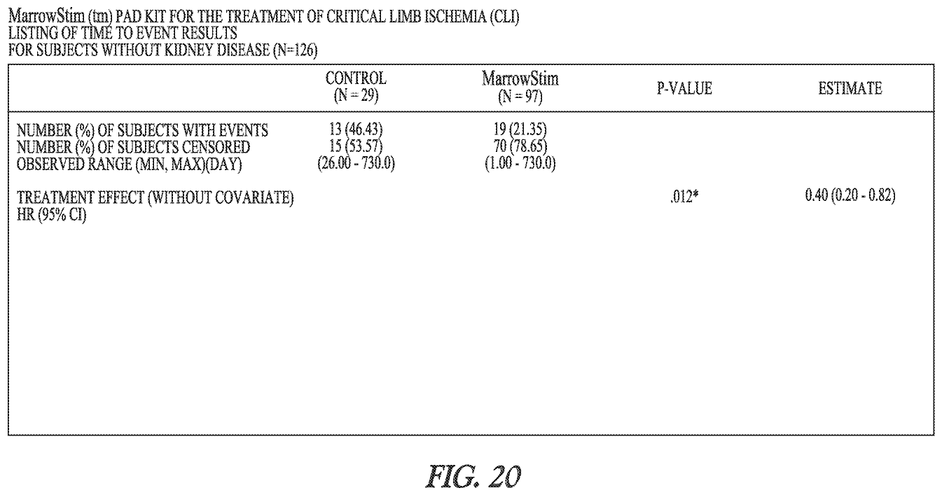

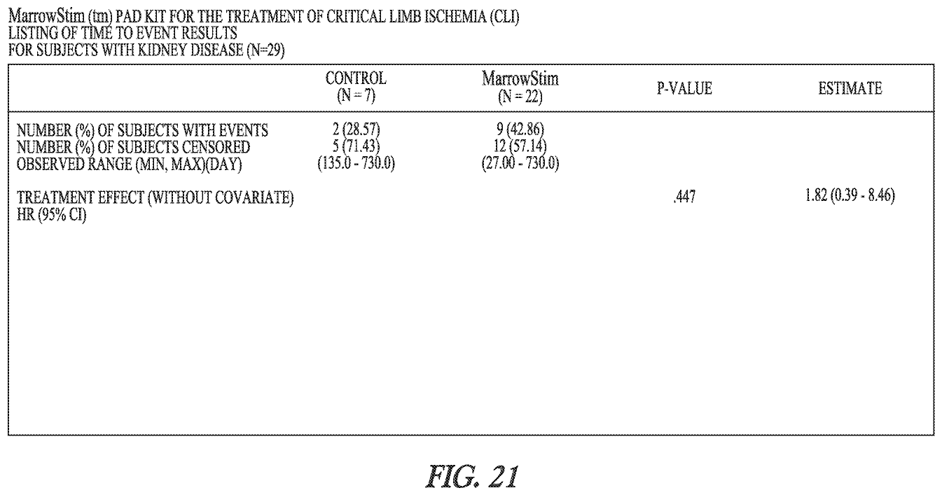

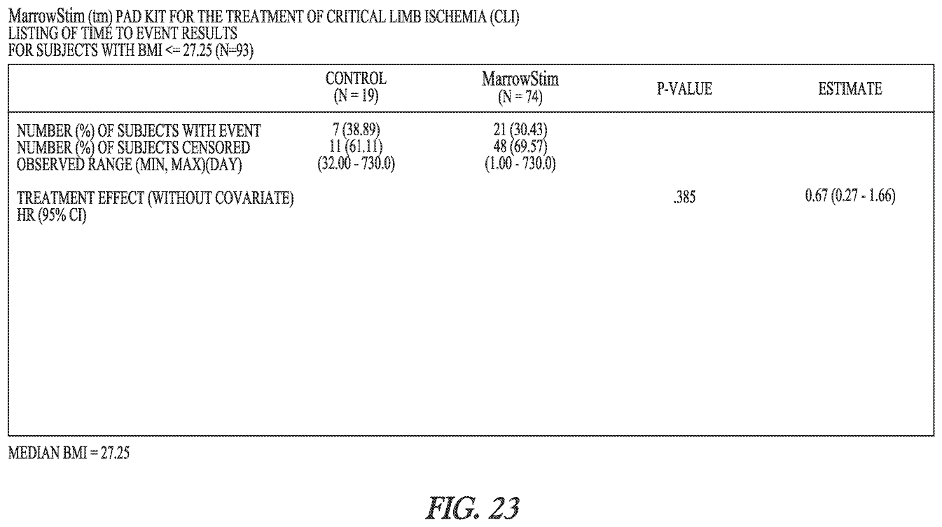

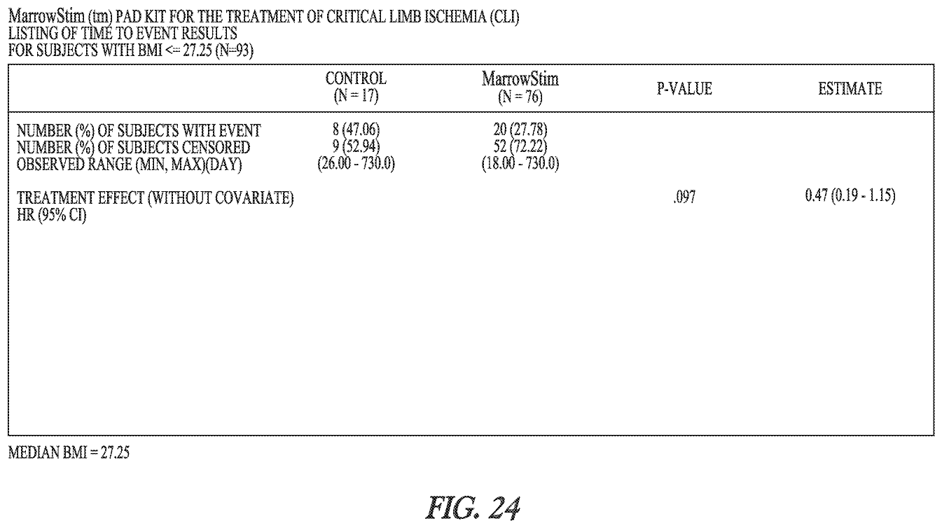

A characteristic of the patient may be a demographic characteristic, a disease history or a symptom. The patient characteristics may be any combination of such characteristics and may further include characteristics external to, but descriptive of, the patient, e.g., such as patient location, hospital or prognosis. Some examples of patient characteristics are listed in FIGS. 1A-34 as covariates. Patient characteristics include, but are not limited to, history of diabetes, a history of kidney disease, history of stroke, history of transient ischemic attack, history of myocardial infarction, history of cardiac arrhythmia, history of congestive heart failure, history of deep vein thrombosis (DVT), a history of amputation, Rutherford severity, sex, ethnicity, age, HbA1c status, ambulatory status, number of wounds, size of wounds, and any combination thereof. Patient characteristics may be each individually defined. A characteristic of the patient may further be patient atherosclerotic disease burden, which may be further defined as, e.g., a combination of any of history of stroke, history of transient ischemic attack, history of myocardial infarction, history of cardiac arrhythmia, history of congestive heart failure, and history of DVT. Another example of a characteristic of a patient is a history of clotting dysfunction, e.g., a history of DVT and cardiac arrhythmia.

Each such characteristic corresponds to a value which is determined and weighted according to influence of the corresponding characteristic on patient outcome in a known population of patients suffering from critical limb ischemia treated with concentrated bone marrow nucleated cells. The known population of patients may be a clinical population having a known treatment outcome or it may be current population of patients having a known treatment outcome from treating of CLI with cBMNC. In some examples, the known population of patients is a clinical trial population.

In various examples, the value is a coefficient determined by multivariate analysis of a population of patients suffering from critical limb ischemia treated with concentrated bone marrow nucleated cells. For example, the values may correspond to values in FIGS. 1-34 provided herein.

Each such value can be used to calculate a patient risk score which can be compared to a pre-determined, known risk score correspond to the patient population from which the values were obtained. Comparison can be used to provide a relative risk, e.g., higher or lower risk, or can be used to compare to the actual, known patient outcome of the treated patient population to determine an estimated chance of adverse outcome, e.g., a <5%, <10%, <20%, <30%, <40% or 50% or great chance of amputation, death, or either. Treatment failure, or adverse patient outcome can be, but is not limited to, amputation of a limb or death.

The determined risk thus corresponds to the risk adverse patient outcome, risk of amputation, risk of death, or any combination thereof, from treating a patient suffering from critical limb ischemia with concentrated bone marrow nucleated cells.

In various examples, the concentrated bone marrow nucleated cells (cBMNC) may be aspirate harvested from bone marrow and concentrated. The cBMNC may be autologous cells obtained from the patient being treated. The cBMNC may be cells harvested and concentrated via a MarrowStim™ device. The cBMNC may be administered by intramuscular injection but is not limited to be administered to the patient by any particular method. For example, the cBMNC may be administered directly to the symptomatic area of the body, e.g., a limb, or it may be administered intravenously or intraarterially, e.g., upstream from the symptomatic area of the body.

The method of the present invention also may include, for example, treating a patient suffering from CLI after determining the risk of treating said patient with cBMNC. The treatment method may involve treatment with cBMNC or it may not. That is, the method in some examples may include identifying a high associated with cBMNC treatment and further comprise administration of a different CLI therapy, i.e., other than cBMNC.

The present invention also provides a system. The system may comprise instructions for practicing the above-described method. As a further example, the system may comprise: a computer including at least one processor and a memory device, the memory device including instructions that, when executed by the at least one processor, cause the computer to:
access a database containing one or more value each corresponding to an inputted characteristic of a patient suffering from critical limb ischemia, wherein each value is determined and weighted according to influence of the corresponding characteristic on patient outcome in a population of patients suffering from critical limb ischemia treated with concentrated bone marrow nucleated cells;
calculate from the one or more values a patient risk score;
compare the calculated patient risk score to a pre-determined risk score corresponding to the population of patients;
determine based on the compared risk scores the risk of treating a patient suffering from critical limb ischemia with concentrated bone marrow nucleated cell.

The present invention also provides a machine-readable storage device. The machine-readable storage device may comprise instructions for practicing the above-described method. As a further example, the machine-readable storage device may include instructions that, when executed by a machine, cause the machine to:

access a database containing one or more value each corresponding to an inputted characteristic of a patient suffering from critical limb ischemia, wherein each value is determined and weighted according to influence of the corresponding characteristic on patient outcome in a population of patients suffering from critical limb ischemia treated with concentrated bone marrow nucleated cells;
calculate from the one or more values a patient risk score;

compare the calculated patient risk score to a pre-determined risk score corresponding to the population of patients;

determine based on the compared risk scores the risk of treating a patient suffering from critical limb ischemia with concentrated bone marrow nucleated cell.

Certain examples are described herein as including logic or a number of components, modules, or mechanisms. Modules may constitute either software modules (e.g., code embodied on a machine-readable medium or in a transmission signal) or modules. A module is tangible unit capable of performing certain operations and may be configured or arranged in a certain manner. In examples, one or more computer systems (e.g., a standalone, client or server computer system) or one or more modules of a computer system (e.g., a processor or a group of processors) may be configured by software (e.g., an application or application portion) as a module that operates to perform certain operations as described herein.

In various examples, a module may be implemented mechanically or electronically. For example, a module may comprise dedicated circuitry or logic that is permanently configured (e.g., as a special-purpose processor, such as a field programmable gate array (FPGA) or an application-specific integrated circuit (ASIC)) to perform certain operations. A module may also comprise programmable logic or circuitry (e.g., as encompassed within a general-purpose processor or other programmable processor) that is temporarily configured by software to perform certain operations. It will be appreciated that the decision to implement a module mechanically, in dedicated and permanently configured circuitry, or in temporarily configured circuitry (e.g., configured by software) may be driven by cost and time considerations.

Accordingly, the term “module” can be understood to encompass a tangible entity, such as hardware, that can be that an entity that is physically constructed, permanently configured (e.g., hardwired) or temporarily configured (e.g., programmed) to operate in a certain manner and/or to perform certain operations described herein. Considering examples in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules can provide information to, and receive information from, other modules. Accordingly, the described modules may be regarded as being communicatively coupled. Where multiple of such modules exist contemporaneously, communications may be achieved through signal transmission (e.g., over appropriate circuits and buses) that connect the modules. In examples in which multiple modules are configured or instantiated at different times, communications between such modules may be achieved, for example, through the storage and retrieval of information in memory structures to which the multiple modules have access. For example, one module may perform an operation, and store the output of that operation in a memory device to which it is communicatively coupled. A further module may then, at a later time, access the memory device to retrieve and process the stored output. Modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some examples, comprise processor-implemented modules.

Similarly, the methods described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example examples, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., Application Program Interfaces (APIs).)

Examples may be implemented in digital electronic circuitry, or in computer hardware, firmware, software, or in combinations of them. Examples may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In examples, operations may be performed by one or more programmable processors executing a computer program to perform functions by operating on input data and generating output. Method operations can also be performed by, and apparatus of examples may be implemented as, special purpose logic circuitry, e.g., a field programmable gate array (FPGA) or an application-specific integrated circuit (ASIC).

The computing system can include clients and servers. A client and server are generally remote from each other and typically interact through a communication network. The relationship of client and server arises by virtue of computer programs running on the respective computers and having a client-server relationship to each other. In examples deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above detailed description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the detailed description as examples or embodiments, with each claim standing on its own as a separate example, and it is contemplated that such examples can be combined with each other in various combinations or permutations. The scope of the invention should be determined with reference to the appended claims, along with the full scope of equivalents to which such claims are entitled.

### Examples

### Data Sources

Data was sourced from two protocols, a one-year pivotal protocol (MOBILE), which was extended to two-years, and a continued access protocol.

The similarity of the inclusion and exclusion criteria from the two protocols suggest data from these studies are combinable.

### Covariate Analysis Methodology

The exploration of the treatment effects for different levels of baseline characteristics is explored here for completeness using data combined from the two-year extension and the continued access protocol.

The suitable method for evaluating treatment effects at different levels of covariates is outlined below.

The treatment effects for different levels of a covariate can be properly detected by an interaction analysis (Roback, Goldstein, Rampey, & Wilson, 1994, the contents of which are hereby incorporated by reference herewith). It may not be detected by the simple inclusion of a covariate in a statistical model. The model without an interaction term ay require that the there is no difference between (or among) levels of the covariate in the treatment effect (Enas, Enas, Spradlin, Wilson, & Wiltse, 1990). Sub-group analyses at the different levels of the covariate would be inefficient since they may not use all of the data from the experiment and would, prior to interpretation, require adjustment for multiplicity.

If treatment performance were different for different levels of the covariate factors beyond what would be expected due to chance that would be evident in such a full interaction model. Such a model would, of course, include the primary analysis terms for treatment, diabetes, Rutherford Severity, and combined site all terms that were factors in the randomization. Additionally, it may include a term for the interaction of treatment with the covariate. If the interaction is significant, then we can conclude that the treatment effect for the sub-groups is statistically significantly different between the two and that the treatment is superior in one group compared to another.

It has been well documented that the event rates for the individual treatment groups alone can be biased (Pocock, 2006). So, in a case where only the interventional group is included in the analysis, a biased estimate may be obtained. For example, improvements in clinic procedures or any other effect ordered by time, cannot be ruled out. But when subjects are randomly assigned to intervention or control, then the estimated treatment effect (or difference) is unbiased due to time. One of the reasons this is true is because any chronologically ordered effects are essentially subtracted out. Secondly, estimates from the analysis of the interventional group alone can only be generalized to the population consistent with these specific demographic and baseline characteristics. This is especially true for the sub-group proportions. The proportions in the sample usually do not match the proportions in the general population and the estimated treatment effect is weighted in favor of the over-represented element of the sub-group. So, the conclusions are additionally biased for the general population because the interventional group was not randomly selected from all subjects with Chronical Limb Ischemia.

Attribution of these biased sub-group effects in single-armed analyses are also limited by their complex interpretations. There is an inability to distinguish between the treatment effects within a sub-group and the effects for that sub-group alone. For example, if a statistically significant improvement compared to another sub-group were observed, it could be argued that sub-group has a greater sensitivity and response to the intervention and not a sub-group response. Alternatively, it could be argued for those results without statistical significance that a sub-group with a greater natural history hazard has a greater response to the intervention. It may therefore be difficult to assign a definitive conclusion regarding sub-group effects from single-armed studies alone.

So, the treatment effects for different levels of a covariate are best estimated by an interaction analysis approach. Tests for the interaction between treatment and a blocking factor can be made at the two-sided, alpha level of 0.10.

Subgroup results are provided. If the conclusions conflicted between the interaction conclusion and the subgroup conclusion the result from the interaction analysis was considered superior.

### Results and Discussion

### One-Year Pivotal Study—Analysis of Study Results

In the top line results for the one-year pivotal study, it was concluded that the primary efficacy analysis shows a smaller than one but statistically insignificant point estimate for the treatment effect hazard ratio after adjusting for covariates in the proportional hazards regression model (HR=0.64, p=0.224) of autologous concentrated bone marrow nucleated cells (cBMNC) therapy for Critical limb ischemia (CLI). The associated 95% confidence interval includes unity (95% lower and upper confidence limits=0.31, 1.31). It was also reported that the 52-week event proportion for MarrowStim was numerically smaller than that for the control (20.17% vs. 30.56%).

In view of such positive results, it is unexpected that in certain sub-groups of patients the control group substantially outperformed expectations. The expectation, based on a pre-study, meta-analysis was estimated at 40% (Wilson, 2014). A post-hoc marginal sub-group results provided some evidence of differing treatment effects particularly for the non-diabetics (p=0.016). Additional investigation of sub-group performance was conducted at that time. These unexpected results raise the further question about treatment performance within the levels of the covariate factors and within various patient populations corresponding to covariate subgroups.

One-Year Pivotal Study—Effects of Rutherford Severity and Diabetic Status

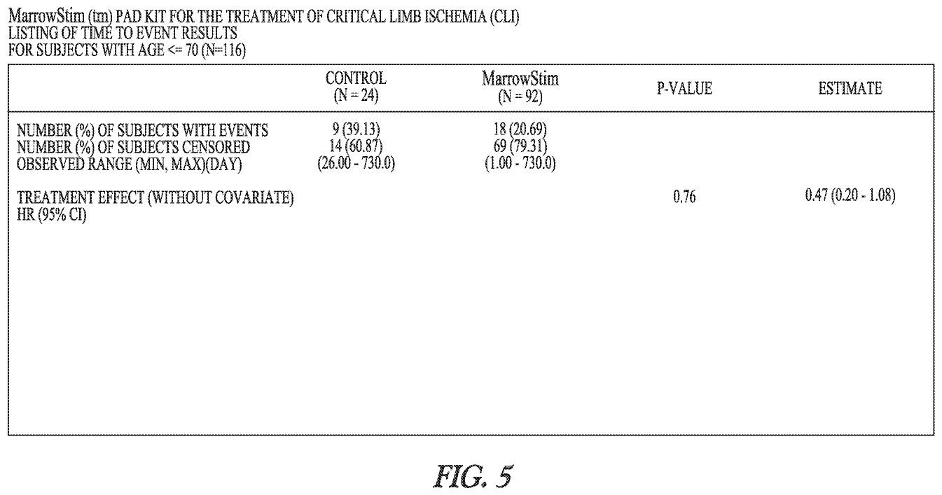

Firstly, the covariate factors included in the randomization (viz., combined site, Rutherford severity, and Diabetic Status; see FIG. 5) were examined. FIG. 5, which includes an upper maximum likelihood (ML) estimates upon which the hazard ratio is based. The interaction between treatment and diabetic status is provided (p=0.073), which is a statistically significant effect. The effect of the interaction between treatment and Rutherford Severity is not significant. Importantly, for this model is the effect of the treatment interaction with combined site. Sites were combined, but the treatment interaction for combined site was removed from the model to assess its impact separately.

One-Year Pivotal Study—Effects of Combined Site

The results of treatment interaction for combined site show interaction between treatment and diabetic status is stable and statistically significant (p=0.074). The effect of the interaction between treatment and Rutherford Severity is again not significant. So, the conclusion of an interaction effect between treatment and diabetic status and no interaction between combined site and Rutherford status can be considered reliable.

One-Year Pivotal Study—Effects of Body Weight, BMI, Smoking and Race

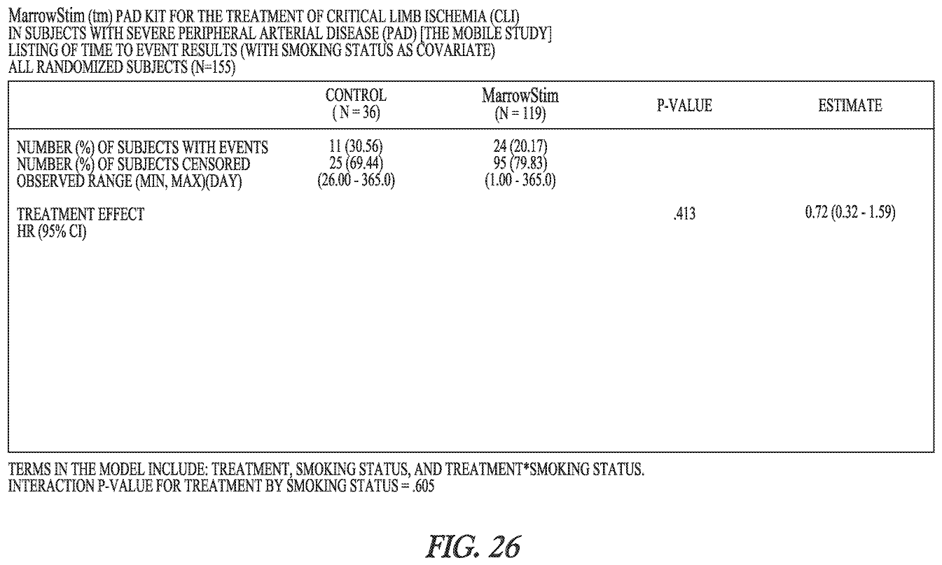

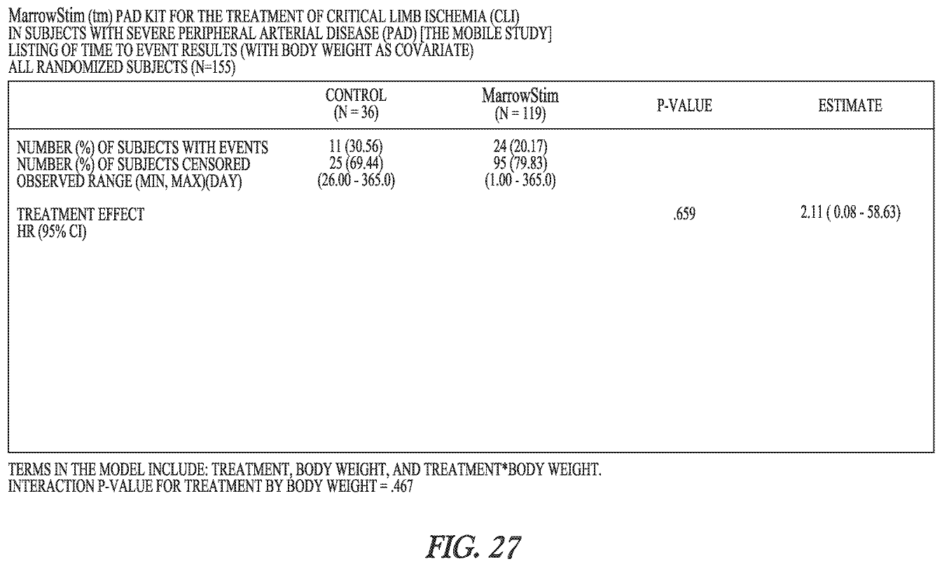

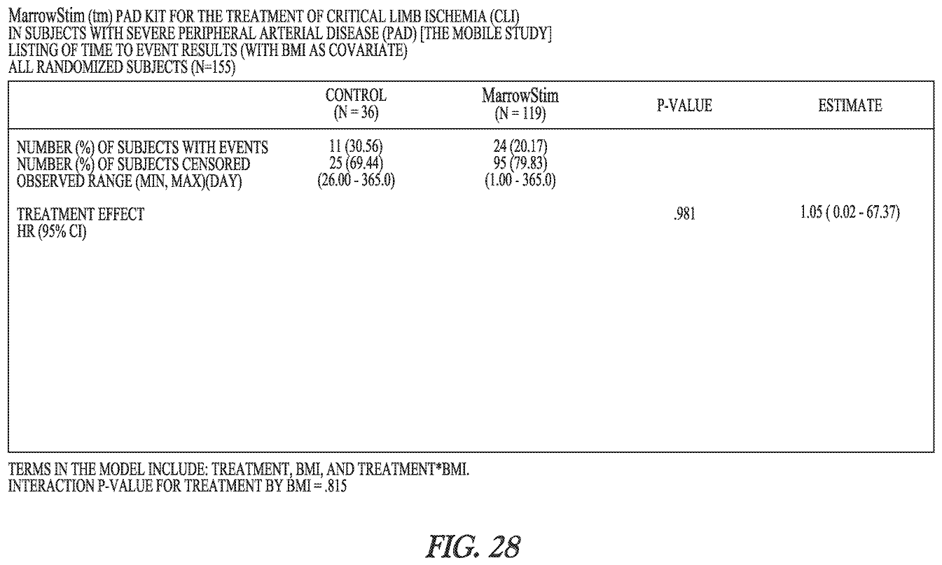

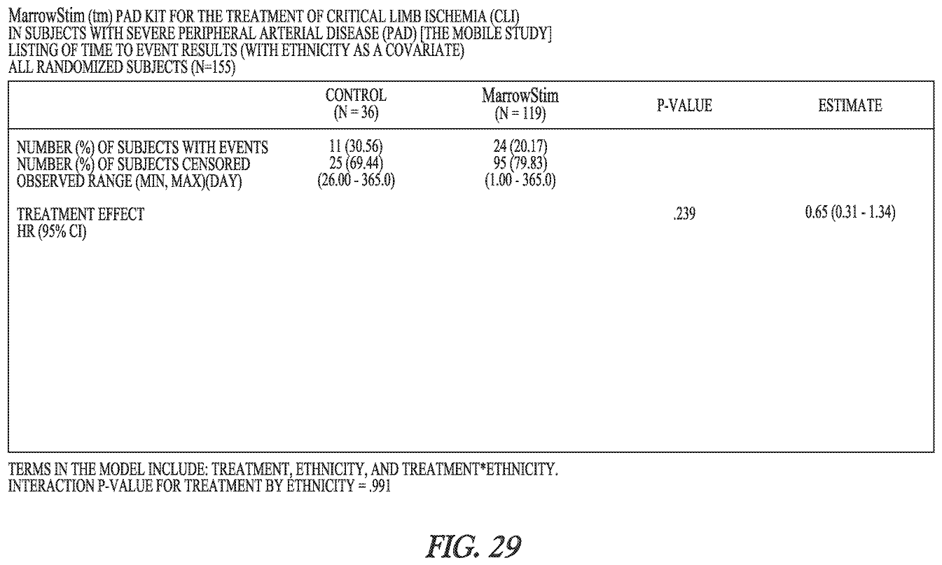

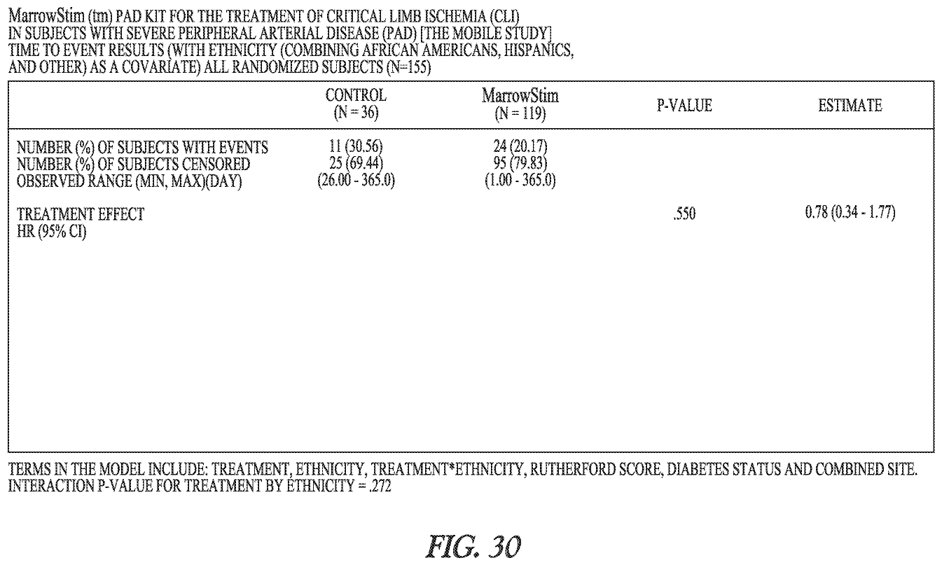

Because such a heterogenous effects of treatment were not expected at least at the 52-week timepoint, many baseline characteristics were therefore not included as a randomization covariate and not included as one of a pre-specified covariate for examination by the full interaction model. However, they were subsequently examined. Those effects included body weight (FIG. 27), body mass index (FIG. 28), and smoking (FIG. 26). The examination of the effects of race were reported (FIG. 29 and FIG. 30) and of particular interest. (Wang et al., 2017)

### One-Year Pivotal Study—Effects of Gender

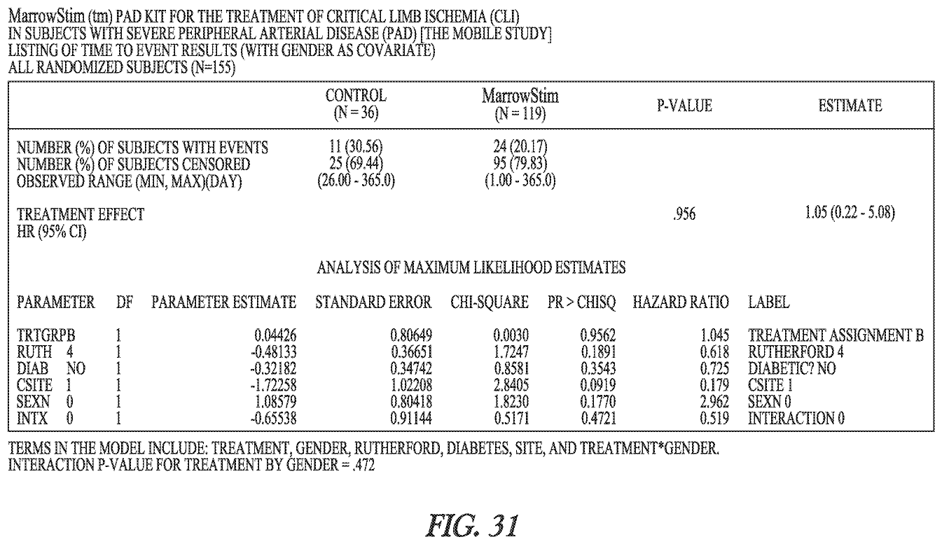

In addition, the effect of gender on the treatment effect was also explored. The results of IM-A are provided in FIG. 31, which includes an upper and lower panel. The ML model converges (results not shown). The upper panel of FIG. 31 provides some summary statistics, the hazard ratio with 95% confidence interval and the lower panel provides the maximum likelihood (ML) estimates upon which the hazard ratio is based. Terms of the model can be seen in the footnotes. Also, in the footnotes the interaction between treatment and the covariate is provided (p=0.472).

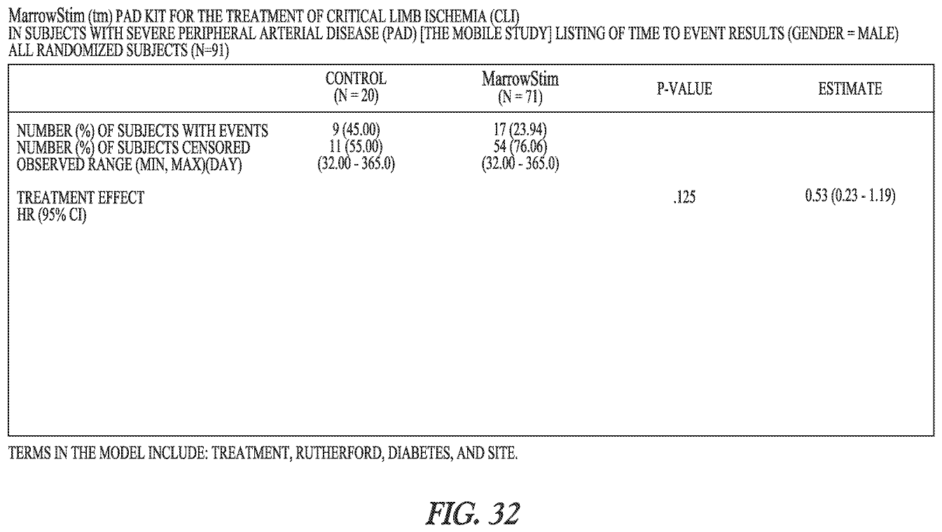

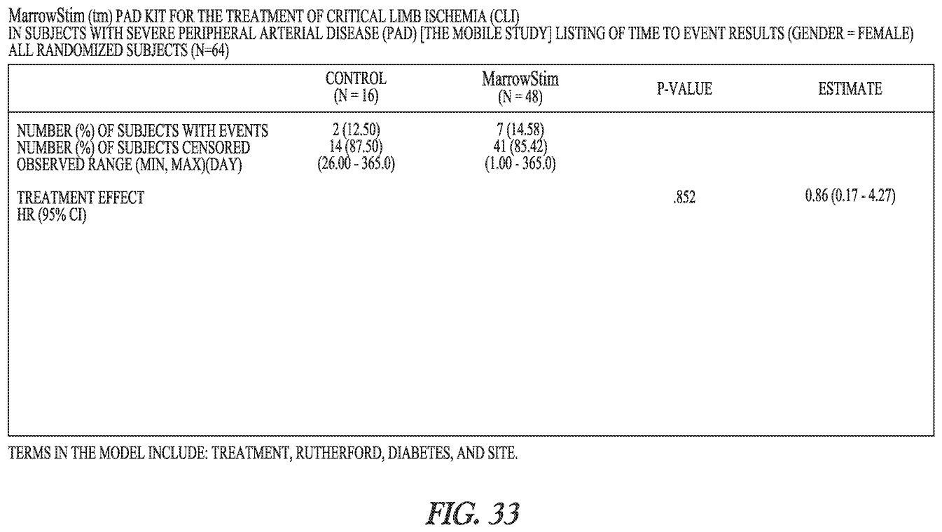

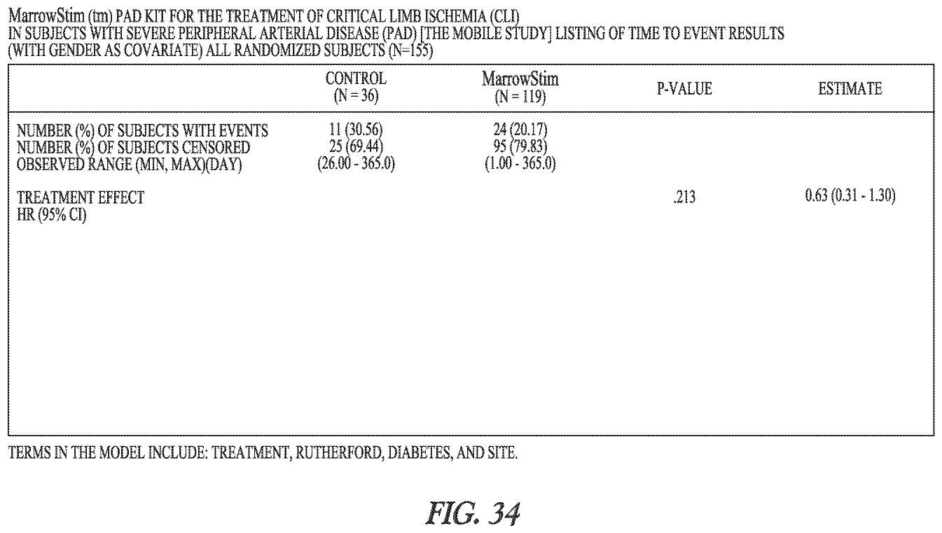

In the upper panel, it can be seen that the hazard ratio estimate, while not significant, reverses direction, is greater than one (HR=1.05) and the width of the confidence interval is more than 4 times wider than for the primary analysis. This suggests that the addition of the covariate and its interaction with the treatment effect have a destabilizing effect on the parameter estimates. The reduced model in FIG. 34 is more stable suggesting the destabilization could be due to a sparsity of data. The width of the confidence interval for the male by-group analyses (FIG. 32) is similar to the primary but not the female (FIG. 33) confidence interval. This suggests that although the model can converge with only two female failures in the control group, it has an overwhelming effect on the variability. In the lower panel it can be seen, that unlike the variance of combined site in the primary model, these variance estimates in the lower panel under the column labeled standard error, appear stable.

The females in the control group, like the diabetics in the primary analysis, substantially outperformed expectations. These results do not suggest that the women with CLI do not respond differently from men to cBMNC therapy.

One-Year Pivotal Study—Summary of Covariate Effects

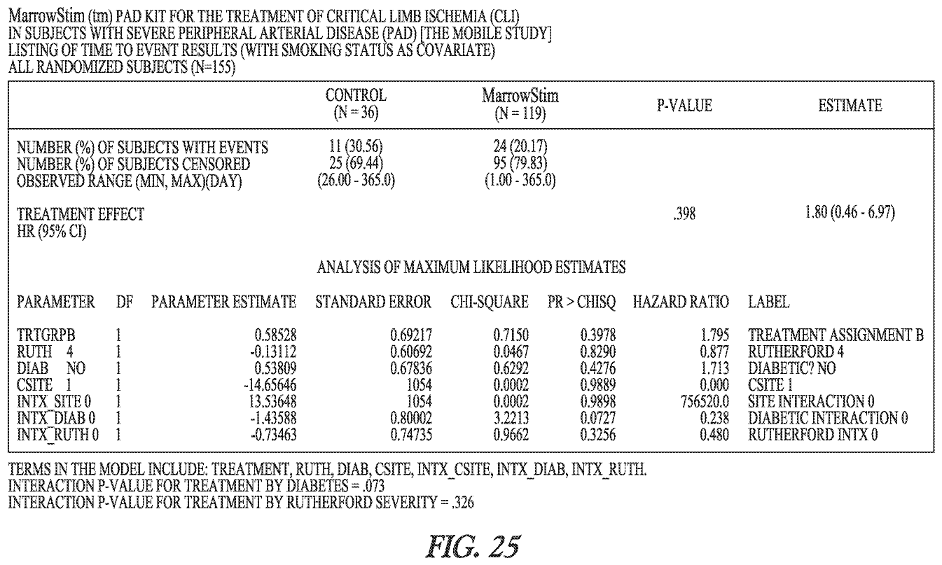

For the one-year pivotal study analyses, only diabetes among these eight covariates, showed a statistically significant difference in treatment effect between levels. The ten tables created to explore these one-year pivotal results and referenced herein are set forth as FIGS. 25-34.

### Combined Extended Pivotal and Continued Access Protocols

### Combinability of Data

Although the similarity of the inclusion and exclusion criteria from the two protocols suggest data from these studies are combinable, differences in recruiting were observed. The ability to combine the data from the two-year pivotal extension and the continued access protocols were also investigated by summarizing the demographics (FIGS. 1A and 1B).

The studies were mostly balanced with the exception being age which was imbalanced with older subjects in the Continued Access protocol (mean+/−standard deviation in years, Continued Access versus Pivotal, n=32, 71.31+/−11.81 vs. n=155, 64.93+/−11.90, p=0.006*). Importantly, the variance for age for the two studies was similar.

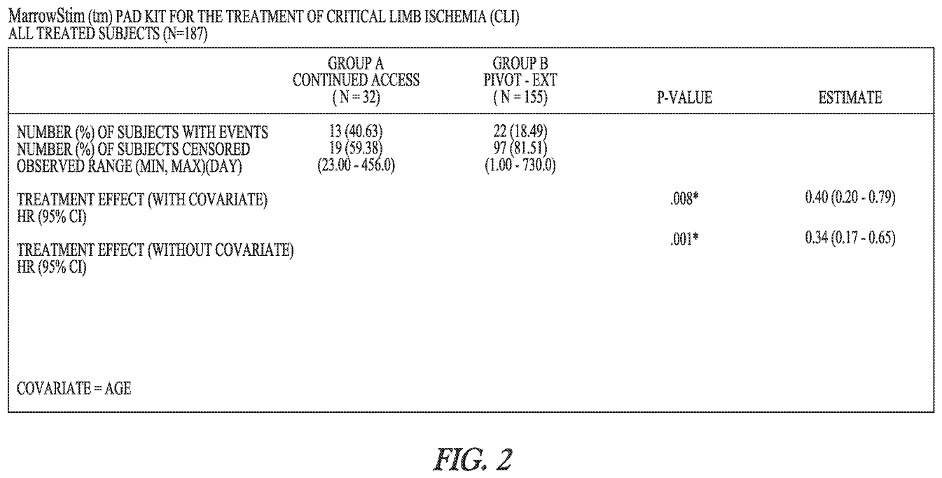

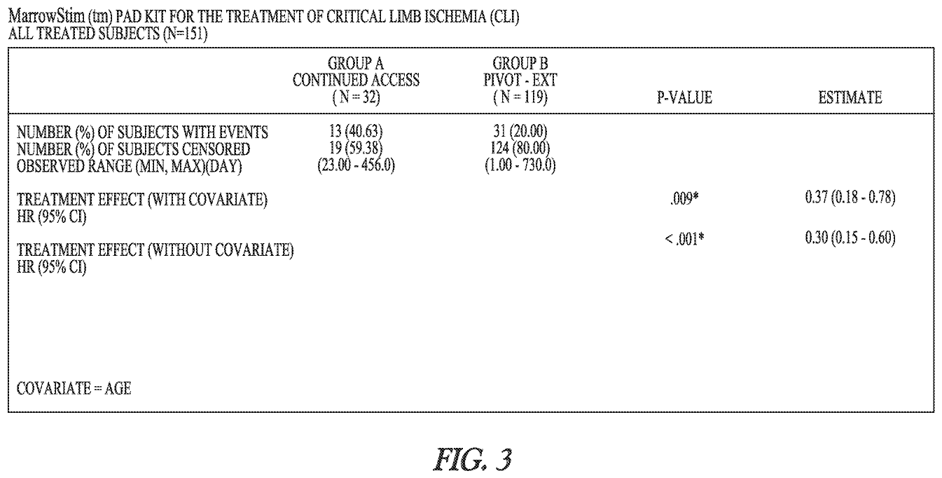

Age is a significant risk factor in all survival analyses and that is reflected the analysis of the events. Combinability was investigated for the time to event analyses for all subjects, unadjusted and adjusted for the imbalance of continuous age (FIG. 2) and for the interventional groups alone also unadjusted and adjusted for continuous age (FIG. 3). In both analyses, including the imbalanced covariate of age widens the confidence interval. All four analyses show a statistically significant difference in the hazard ratio between studies. The studies can possibly be combined with these important findings and caveats for age and hazards.

### Combined Protocol—Effects of Baseline Age

Age at which a subject enters a study is a significant risk factor for survival. Age is imbalanced between the present two studies. So, it is not surprising that there is a difference in hazards between studies, both before and after adjusting for continuous baseline age. To investigate the treatment difference at different age levels, continuous age was dichotomized at 70 and 80 years.

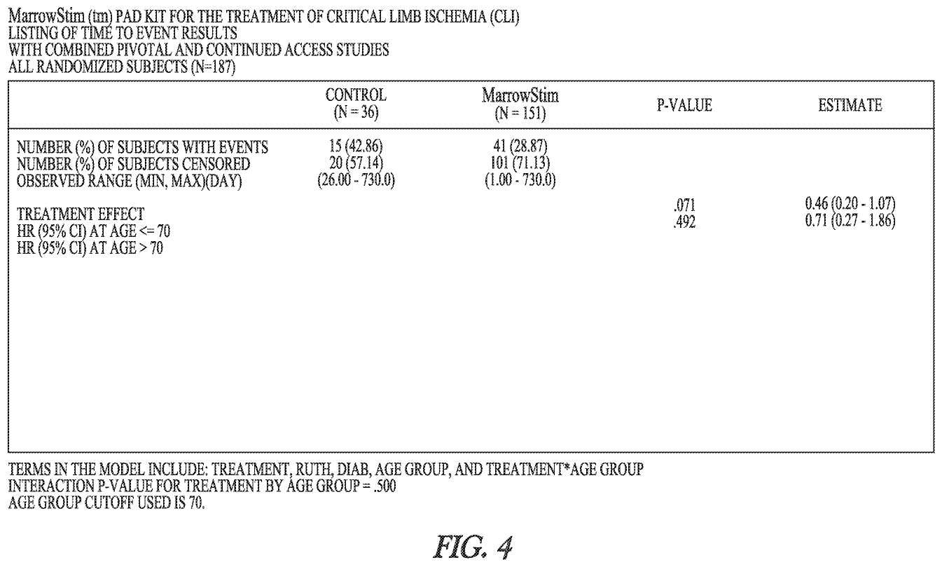

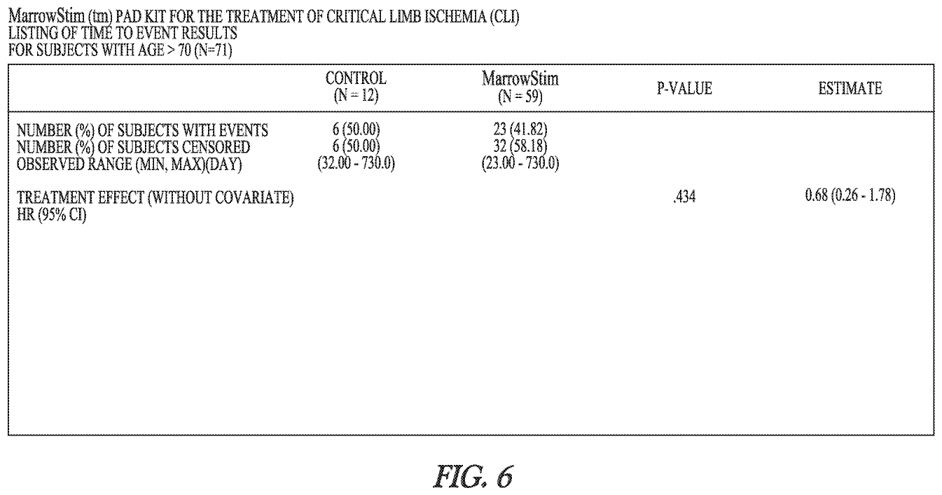

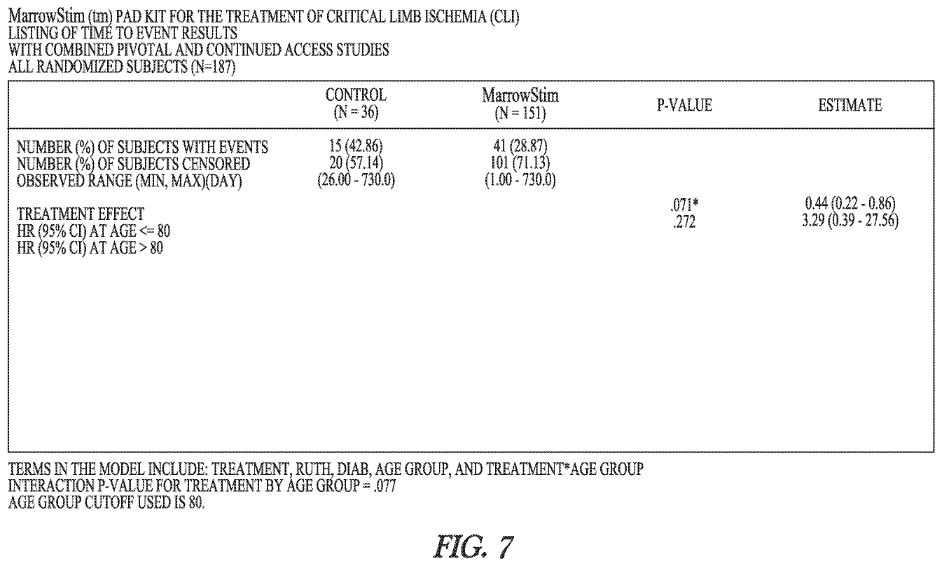

There is no effect of age dichotomized at 70 years on the treatment difference (treatment-by-Age70 interaction p-value=0.500; FIG. 4). Summaries are provided in FIG. 5 and FIG. 6 for the younger and older sub-groups, respectively. However, there is a significant effect of age dichotomized at 80 years on the treatment difference (treatment-by-Age80 interaction p-value=0.077; FIG. 7). The hazard ratio in the younger sub-group is 0.44 (95% confidence interval,

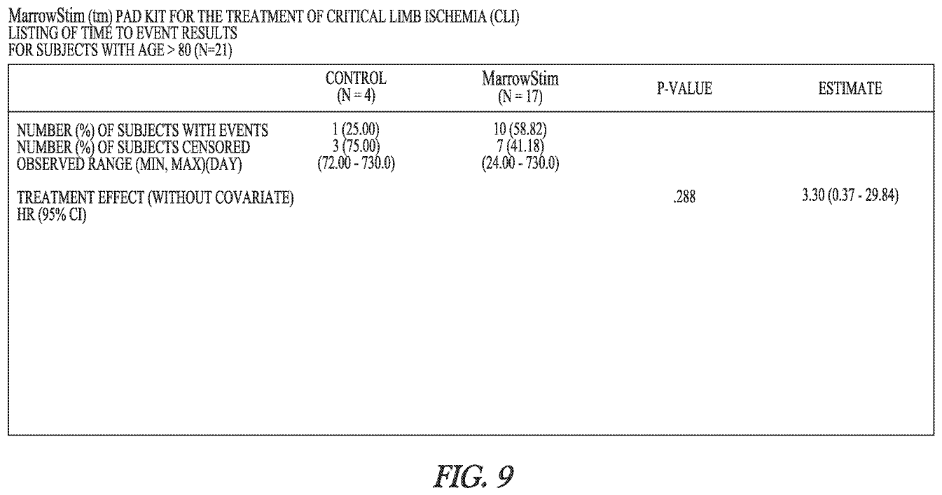

0.22-0.86; p=0.01⁷*). The hazard ratio for the older sub-group is not statistically significant partially due to the wide confidence interval which is a function of the small sample size in the control group (n=4; FIG. 9) and partially due to the large number of events in the interventional group.

### Combined Protocol—Effects of Gender

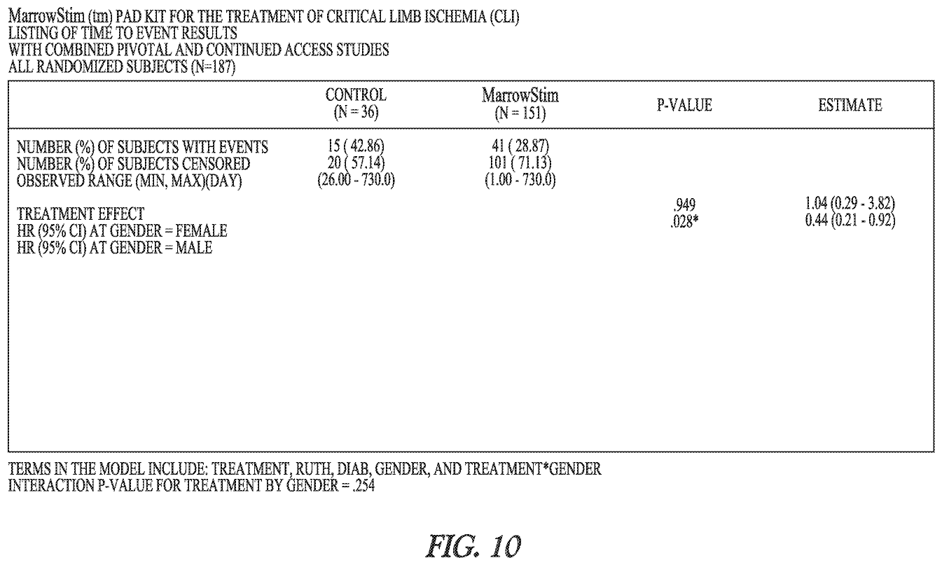

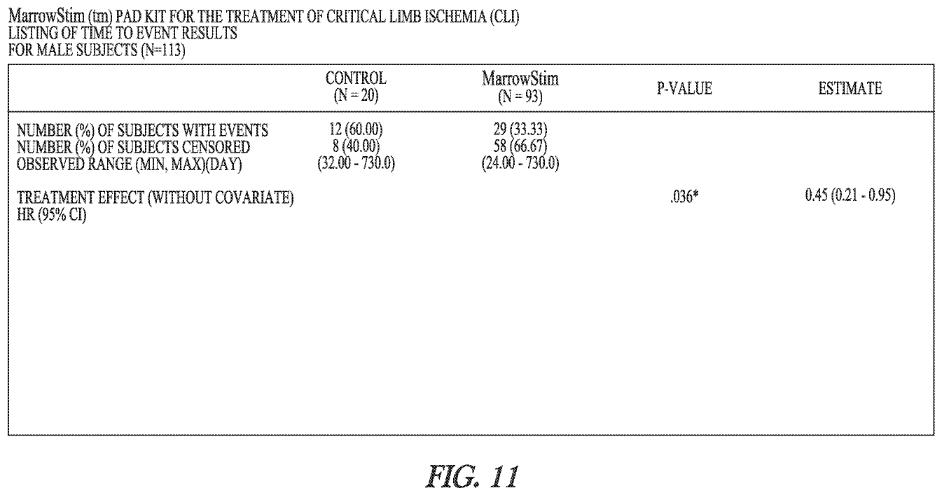

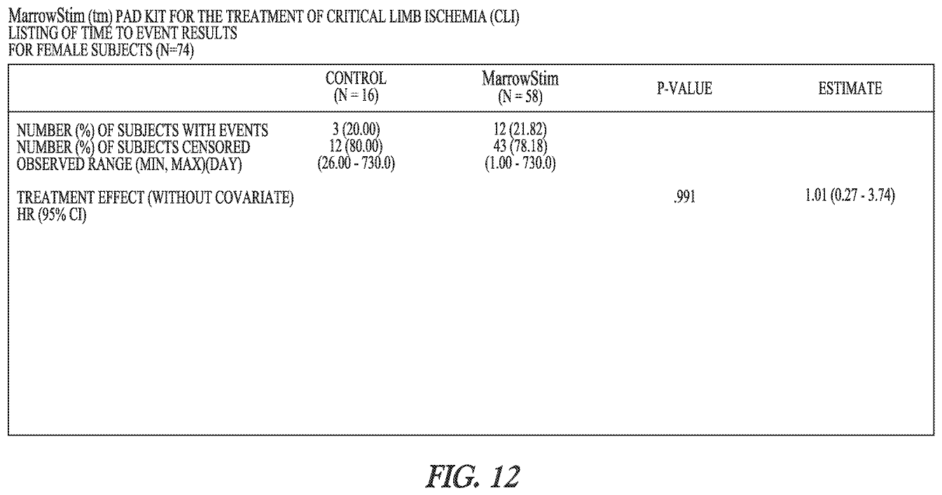

In the combined protocol analysis of gender, fewer females than males (n=74 vs. n=113) participated. The substantially outperformed expectations observed for females during the one-year analyses, remained observable (FIG. 12). However, this observation was not statistically significant and this difference were not greater than would be expected by chance alone. It is true the treatment effect for the male-only sub-group at first appears statistically significant (p-value=0.036; FIG. 11). Although, similar to the results from the one-year pivotal data (See Section 1.2.4 above), the combined protocol exploratory analysis suggests no differences between genders in the treatment effect (treatment-by-gender interaction p-value=0.254; FIG. 10).

### Combined Protocol—Effects of Ethnicity

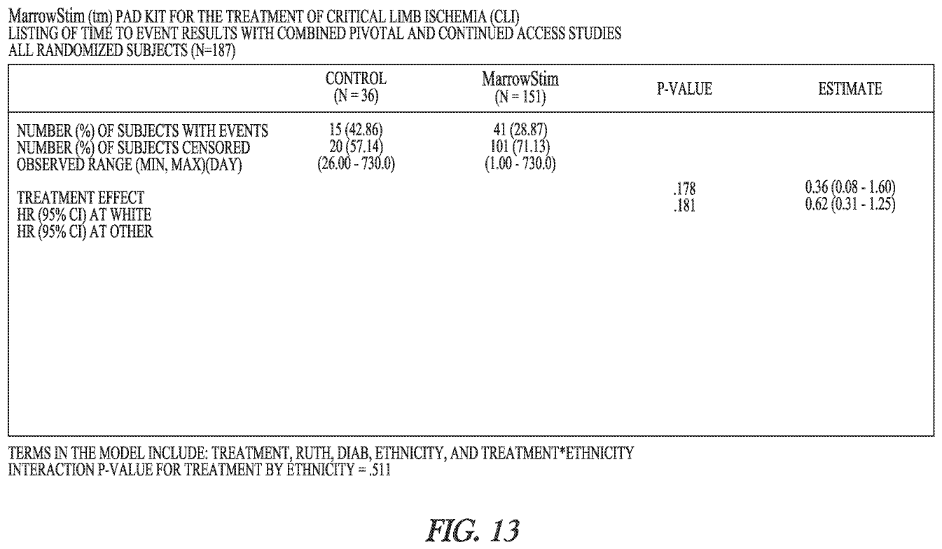

Ethnicity was balanced between studies. There is no effect of ethnicity on the treatment difference (treatment-by-Ethnicity interaction p-value=0.511; FIG. 13).

### Combined Protocol—Effects of Glvcosylated Hemoglobin

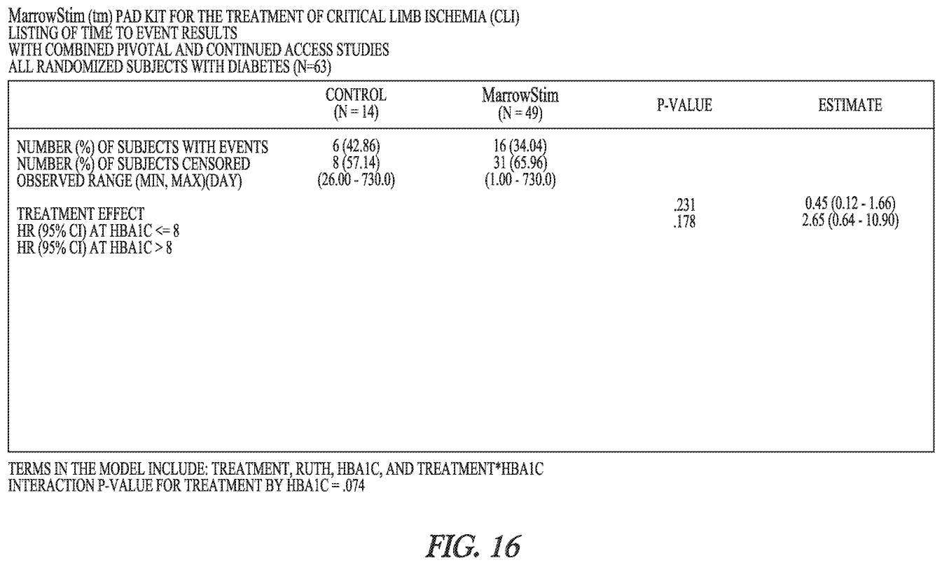

HbA1c was measured in 63 diabetic subjects. This group was dichotomized at the median of 8 into those subjects with results below the median and above the median. Within those subjects, there is a statistically significant effect of glycosylated hemoglobin on the treatment difference (treatment-by-HbA1c category interaction p-value=0.074; FIG. 16). However, due to the small sample size, the hazard ratios for neither the below the median and above the median sub-group, were statistically significant.

### Combined Protocol—Effects of Renal Disease

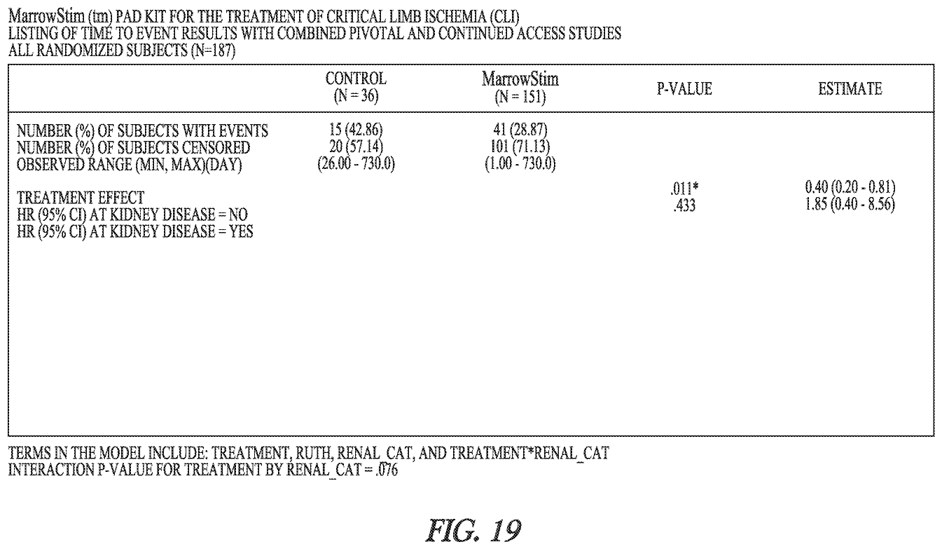

Subjects were categorized by the presence or absence of renal disease at baseline. There is a statistically significant effect of renal disease on the treatment difference (treatment-by-HbA1c category interaction p-value=0.076; FIG. 19). There is a statistically significant treatment effect for those subjects who did not report the presence of renal disease at baseline, HR=0.40 (95% CI=0.20-0.81; p=0.011*). No significant difference was observed for those subjects who reported renal disease.

### Combined Protocol—Effects of Body Mass Index

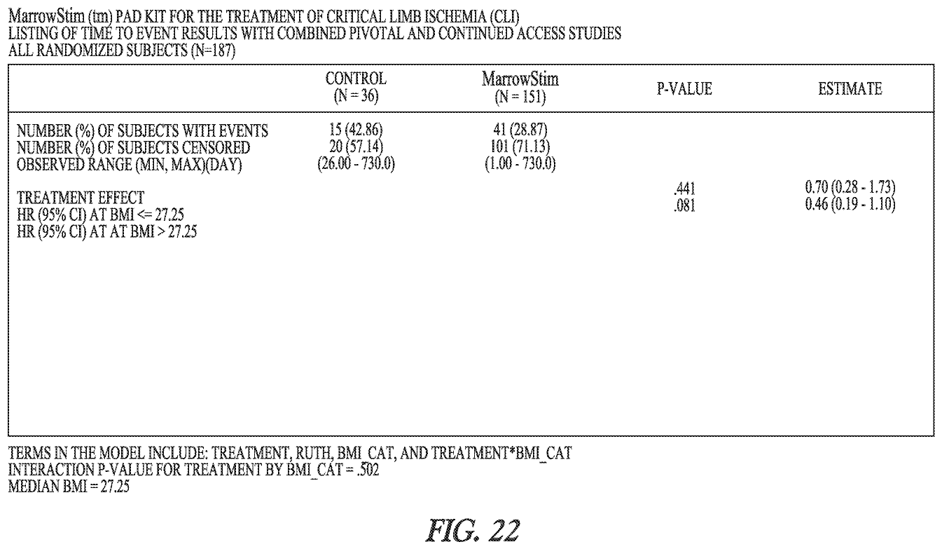

Body Mass Index (BMI) was balanced between studies. The results were categorized as results below the median and above the median. The median was 27.25 kg/m². There is no effect of BMI on the treatment difference (treatment-by-BMI interaction p-value=0.511; FIG. 22).

HbA1c was measured in 63 diabetic subjects. This group was dichotomized at the median of 8 into those subjects with results below the median and above the median. Within those subjects, there is a statistically significant effect of glycosylated hemoglobin on the treatment difference (treatment-by-HbA1c category interaction p-value=0.502; FIG. 19).

### Combined Protocol—Summary of Covariate Effects

For the combined results from the two-year extension and the continued access protocol, the risk factors for treatment failure, identified are those subjects who are diabetic, older than 80 years and have renal disease. Alternatively, these data suggest that the subjects with the best change to do well are those who are non-diabetic, less than 70 and without renal disease. These data suggest the covariates not associated with risk for treatment failure are Rutherford Severity, Gender, Ethnicity, Glycosylated Hemoglobin, and Body Mass Index. Accordingly, the covariates associated with risk correspond to patient characteristics which may be advantageously used to determine a risk score for treatment of a patient suffering from CLI with cBMNC.

### Example of Risk Prediction Using Risk Numbers

The risk of adverse event of using concentrated bone marrow nucleated cells can be predicted by Risk Number Prediction. Patient characteristics, namely, age, Rutherford scores, diabetes status and corresponding endpoint data were used to generate a risk score for each patient. Patients were assigned cumulative points based on their underlying characteristics. Specifically, diabetic status (No=0, Yes=1), Rutherford Category (R4=0, R5=1), and age (<70 years=0, 70-79 years=1, and 80 years or greater=2). The scores for each patient were summed and compared to the associated event rate for the underlying patients. Tables 1-3 show example calculated Risk Scores for patients in the combined protocols (Table 1), the extended Pivotal protocol (Table 2) and the Continued Access protocol (Table 3). As shown in each of Tables 1-3, Risk score correlates with Event Rate. Risk scores can be further interpreted to determine high risk (e.g., a risk score of 4), medium risk (e.g., a risk score of 1-3) and low risk (e.g., a risk score of 0), based on how the risk scores stratify within a population of patients.

TABLE 1
Combined Protocols

| Risk Score | n | % | Event Rate |
|---|---|---|---|
| 4 | 8 | 5.1% | 87.5% |
| 3 | 24 | 15.3% | 29.2% |
| 2 | 39 | 24.8% | 25.6% |
| 1 | 55 | 35.0% | 18.2% |
| 0 | 31 | 19.7% | 6.4% |
| Total | 157 | 100.0% | — |

TABLE 2
Pivotal Protocol

| Risk Score | n | % | Event Rate |
|---|---|---|---|
| 4 | 4 | 3.4% | 100.0% |
| 3 | 16 | 13.4% | 31.3% |
| 2 | 26 | 21.8% | 19.2% |
| 1 | 46 | 38.7% | 17.4% |
| 0 | 27 | 22.7% | 7.4% |
| Total | 119 | 100.0% | — |

TABLE 3
Continued Access Protocol

| Risk Score | n | % | Event Rate |
|---|---|---|---|
| 4 | 4 | 10.5% | 75.0% |
| 3 | 8 | 21.1% | 25.0% |
| 2 | 13 | 34.2% | 38.5% |
| 1 | 9 | 23.7% | 22.2% |
| 0 | 4 | 10.5% | 0.0% |
| Total | 38 | 100.0% | — |

The same process can be used to tally atherosclerotic disease burden number, e.g., (1 point for each of a history of stroke, myocardial infarction, deep vein thrombosis, cardiac arrhythmia, etc) which may be further used to determine high, medium and low atherosclerotic disease burden, and used in a subsequent Risk Number prediction. To further exemplify risk score prediction using risk numbers, the same process can use weighted points, e.g., diabetic status (no=0, yes=2) to emphasize the influence of a given patient characteristic on risk. Lastly, additionally, per the results of the multivariate analysis above, Risk can be advantageously predicted based on diabetes, age and renal disease.

Example of Risk Score Prediction Using Coefficients from Multivariate Analysis
Risk Score may also be generated in a similar manner as risk as above, except that rather than using additive whole number points for each covariate, coefficients from multivariate analysis are used. As in the examples above, it may be advantageous to use coefficients corresponding to relevant patient characteristics, e.g., diabetes status, renal disease, and age.

### REFERENCES

Enas, G. G., Enas, N. H., Spradlin, C. T., Wilson, M. G., & Wiltse, C. G. (1990). Baseline Comparability in Clinical Trials: Prevention of “Poststudy Anxiety.” Drug Information Journal, 24(3), 541-548. http://doi.org/10.1177/009286159002400312
Pocock, S. J. (2006). Practice between treatments. BMJ, 332(May), 1256-1258.
Roback, P. J., Goldstein, D. J., Rampey, A. H., & Wilson, M. G. (1994). Baseline Predictors of success when comparing two treatments. Obestity Research, 2(4), 337-347.
Wang, S. K., Green, L. A., Gutwein, A. R., Drucker, N. A., Babbey, C. M., Gupta, A. K., . . . Murphy, M. P. (2017). Ethnic Minorities with Critical Limb Ischemia Derive Equal Amputation Risk Reduction from Autologous Cell Therapy Compared to Caucasians. Journal of Vascular Surgery.
Wilson, M. G. (2014). Statistical Analysis Plan for MarrowStim study. Warsaw, Ind.

The claimed invention is:

1. A method for determining risk of treating a patient suffering from critical limb ischemia with concentrated bone marrow nucleated cells, comprising:
calculating a patient risk score using a machine from one or more values each corresponding to a characteristic of the patient, wherein
each of the one or more values is determined and weighted according to influence of the characteristic on patient outcome in a population of patients suffering from critical limb ischemia treated with concentrated bone marrow nucleated cells;
comparing the calculated patient risk score to a pre-determined risk score corresponding to the population of patients; and
determining from the comparing the calculated patient risk score to the pre-determined risk score a risk of treating a patient suffering from critical limb ischemia with concentrated bone marrow nucleated cells.

2. The method of claim 1, wherein each characteristic is, individually, a demographic characteristic, a disease history, a symptom, or any combination thereof.

3. The method of claim 1, wherein each characteristic is, individually, a history of diabetes, a history of kidney disease, history of stroke, history of transient ischemic attack, history of myocardial infarction, history of cardiac arrhythmia, history of congestive heart failure, history of DVT, a history of amputation, Rutherford category, sex, ethnicity, age, HbA1c status, ambulatory status, number of wounds, size of wounds, or any combination thereof.

4. The method of claim 1, wherein each of the one values corresponds to a characteristic which is a history of diabetes, a history of renal disease, Rutherford score, or age.

5. The method of claim 1, wherein the patient risk score is calculated, at least in part, based on patient age.

6. The method of claim 1, wherein the patient risk score is calculated, at least in part, based on the patient history of diabetes.

7. The method of claim 1, wherein the patient risk score is calculated, at least in part, based on the patient history of renal disease.

8. The method of claim 1, wherein the patient risk score is calculated, at least in part, based on the patient atherosclerotic disease burden.

9. The method of claim 1, wherein each of the one or more values is a coefficient determined by multivariate analysis of a population of patients suffering from critical limb ischemia treated with concentrated bone marrow nucleated cells.

10. The method of claim 1, wherein patient outcome is quantified in terms of time to amputation or death.

11. The method of claim 1, wherein the concentrated bone marrow nucleated cells are autologous cells harvested and concentrated via a MarrowStim device.

12. The method of claim 1, further comprising treating the patient with a treatment expected to have the same or lower risk compared to the determined risk.

13. The method of claim 1, further comprising treating the patient with the concentrated bone marrow nucleated cells.

14. A system, comprising:
a computer including at least one processor and a memory device, the memory device including instructions that, when executed by the at least one processor, cause the computer to:
access a database containing one or more values each corresponding to an inputted characteristic of a patient suffering from critical limb ischemia, wherein each of the one or more values is determined and weighted according to influence of the inputted characteristic on patient outcome in a population of patients suffering from critical limb ischemia treated with concentrated bone marrow nucleated cells;
calculate from the one or more values a patient risk score;
compare the calculated patient risk score to a pre-determined risk score corresponding to the population of patients;
determine based on the compared risk scores the risk of treating a patient suffering from critical limb ischemia with concentrated bone marrow nucleated cells.

15. A machine-readable storage device including instructions that, when executed by a machine, cause the machine to:
access a database containing one or more values each corresponding to an inputted characteristic of a patient suffering from critical limb ischemia, wherein each of the one or more values is determined and weighted according to influence of the inputted characteristic on patient outcome in a population of patients suffering from critical limb ischemia treated with concentrated bone marrow nucleated cells;
calculate from the one or more values a patient risk score;
compare the calculated patient risk score to a pre-determined risk score corresponding to the population of patients;
determine based on the compared risk scores the risk of treating a patient suffering from critical limb ischemia with concentrated bone marrow nucleated cells.

16. The device of claim 15, wherein the inputted characteristic is, individually, a demographic characteristic, a disease history, a symptom, or any combination thereof.

17. The device of claim 15, wherein inputted characteristic is, individually, a history of diabetes, a history of kidney disease, history of stroke, history of transient ischemic attack, history of myocardial infarction, history of cardiac arrhythmia, history of congestive heart failure, history of DVT, a history of amputation, Rutherford category, sex, ethnicity, age, HbA1c status, ambulatory status, number of wounds, size of wounds, or any combination thereof.

18. The device of claim 15, wherein the one or more values corresponds to a characteristic which is a history of diabetes, a history of renal disease, Rutherford score, or age.

19. The device of claim 15, wherein the patient risk score is calculated, at least in part, based on at least one of patient age, patient history of diabetes, patient history of renal disease, or patient atherosclerotic disease burden.

20. The device of claim 15, wherein each of the one or more values is a coefficient determined by multivariate analysis of a population of patients suffering from critical limb ischemia treated with concentrated bone marrow nucleated cells.
